(19) United States
(12) Patent Application Publication
(10) Pub. No.: US 2022/0090022 A1
(43) Pub. Date: Mar. 24, 2022

(54) GENERATING INDUCED PLURIPOTENT STEM CELLS

(71) Applicant: Board of Regents, The University of Texas System, Austin, TX (US)

(72) Inventor: Thomas Boland, El Paso, TX (US)

(21) Appl. No.: 17/173,887

(22) Filed: Feb. 11, 2021

Related U.S. Application Data
(60) Provisional application No. 62/972,948, filed on Feb. 11, 2020.

(51) Int. Cl. C12N 5/074; C12M 3/00

(52) U.S. Cl. CPC C12N 5/0696; C12N 2506/45; C12M 21/08

(57) ABSTRACT

Procedures for generating induced pluripotent stem cells include applying stress to a somatic cell to produce strain that induces the somatic cell to become a pluripotent stem cell without a need for transfection. Generating induced pluripotent stem cells can include forming a droplet that surrounds a somatic cell; and applying mechanical stress to the somatic cell to produce mechanical strain that induces the somatic cell to become a pluripotent stem cell without transfection, where forming the droplet includes thermal inkjet printing. This can also include printing the pluripotent stem cell on a substrate and incubating the pluripotent stem cell. In that case, forming an aggregate composing the pluripotent stem cell can include, after printing and before incubating, positioning the substrate above the pluripotent stem cell whereby gravity exerts a force on the pluripotent stem cell away from the substrate.

### CROSS REFERENCE TO RELATED APPLICATION

This application is a utility conversion of, and claims priority to, U.S. Ser. No. 62/972,948, filed Feb. 11, 2020, the contents of which are incorporated herein by reference in their entirety for all purposes.

### BACKGROUND INFORMATION

### 1. Field

The present invention relates generally to the field of pluripotent stem cells. More particularly, it concerns generating induced pluripotent stem cells.

### 2. Background

Somatic cells can be induced to become pluripotent stem cells. Okita, K., Ichisaka, T, & Yamanaka, S. Generation of germline-competent induced pluripotent stem cells. Nature 448, 313-317 (2007) (https://doi.org/10.1038/nature05934). Although these cells could be derived from an individual for whom the stem cell therapy is designed and there would be no immune rejection of these cells, there are concerns with the approach by Shinya Yamanaka. These cells need to be transfected with 3 or more genes to become induced and there are concerns about long term safety of using these transfected cells in patients.

Heretofore, the requirement(s) of generating induced pluripotent stem cells from somatic cells without transfection referred to above has not been fully met. In view of the foregoing, there is a need in the art for a solution that solves this problem.

### SUMMARY

There is a need for the following embodiments of the present disclosure. Of course, the present disclosure is not limited to these embodiments. An overall goal of embodiments of the present disclosure is to generate induced pluripotent stem cells.

An illustrative embodiment of the present disclosure provides a method of generating induced pluripotent stem cells, comprising applying mechanical stress to a somatic cell to produce mechanical strain that induces the somatic cell to become a pluripotent stem cell.

An illustrative embodiment of the present disclosure provides a method of generating induced pluripotent stem cells, comprising forming a droplet that surrounds a somatic cell; and applying mechanical stress to the somatic cell to produce mechanical strain that induces the somatic cell to become a pluripotent stem cell without transfection. Optionally, the method can also include printing the pluripotent stem cell on a substrate and incubating the pluripotent stem cell, where after printing and before incubating, positioning the substrate above the pluripotent stem cell whereby gravity exerts a force on the pluripotent stem cell away from the substrate.

An illustrative embodiment of the present disclosure provides a method of generating induced pluripotent stem cells, comprising forming a droplet that surrounds a somatic cell; and applying mechanical stress to the somatic cell to produce mechanical strain that induces the somatic cell to become a pluripotent stem cell without transfection, wherein forming the droplet includes thermal inkjet printing.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D depict a printing device (FIG. 1A) and schematics of a firing chamber holding the cells (FIGS. 1B-1D) in accordance with an illustrative embodiment.

FIGS. 2A-2B depict bioprinted fibroblasts expressing Oct-4 24 hours after bioprinting in accordance with an illustrative embodiment.

FIG. 3 depicts cells stained with a red fluorescent anti-nanog monoclonal antibody in accordance with an illustrative embodiment.

FIG. 4 depicts identical sample as FIG. 3 when cells are stained with anti-Oct-4 monoclonal antibody (green), anti-nano monoclonal antibody (red) and cell nuclei are stained with DAPI (blue) in accordance with an illustrative embodiment.

FIGS. 5A-5D depict cells stained with FIG. 5A anti-Sox2 monoclonal antibody (red), FIG. 5B anti-Oct-4 monoclonal antibody (green), FIG. 5C nucleus with DAPI (blue) and FIG. 5D overlay of A, B and C in accordance with an illustrative embodiment.

FIGS. 6A-6B depict schematics of the necessary elements to induce fibroblasts into expressing stem cell markers: orifice, and drop formation (orifice is optional) in accordance with an illustrative embodiment.

FIG. 7 depicts morphology of printed cells grown in cardiomyocyte differentiation medium. Cells appear long and square in shape which is common for adult cardiomyocytes in accordance with an illustrative embodiment

FIG. 8 depicts bioprinted fibroblasts expressing Oct-4 approximately 24 hours after bioprinting in accordance with an illustrative embodiment.

FIGS. 9A-9D depict cells printed into wells (no aggregates are shown) FIG. 9A Nuclei, FIG. 9B Oct-4, FIG. 9C Nanog and FIG. 9D overlay of A, B and C in accordance with an illustrative embodiment.

FIGS. 10A-10D depict cells printed into wells (no aggregates are shown) FIG. 10A Nuclei, FIG. 10B Oct-4, FIG. 10C Sox-2 and FIG. 10D overlay of A, B and C in accordance with an illustrative embodiment.

FIGS. 11A-11B depict cells printed into wells and cultured in a hanging drop (aggregates) in accordance with an illustrative embodiment. FIG. 11A is an overlay of cell nuclei (blue), Oct-4 (green) and Sox-2 (red). FIG. 11B is an overlay of cell nuclei (blue), Oct-4 (green) and nanog (red).

### DETAILED DESCRIPTION

Embodiments presented in the present disclosure and the various features and advantageous details thereof are explained more fully with reference to the nonlimiting embodiments that are illustrated in the accompanying drawings and detailed in the following description. Descriptions of well-known materials, techniques, components and equipment are omitted so as not to unnecessarily obscure the embodiments of the present disclosure in detail. It should be understood, however, that the detailed description and the specific examples are given by way of illustration only and not by way of limitation. Various substitutions, modifications, additions and/or rearrangements within the scope of the underlying inventive concept will become apparent to those skilled in the art from this disclosure.

Embodiments of this disclosure can include an automated device that enables manufacture by generation of induced pluripotent stem cells from somatic cells. These induced stem cells have potentially less long-term concerns compared to transfected cells.

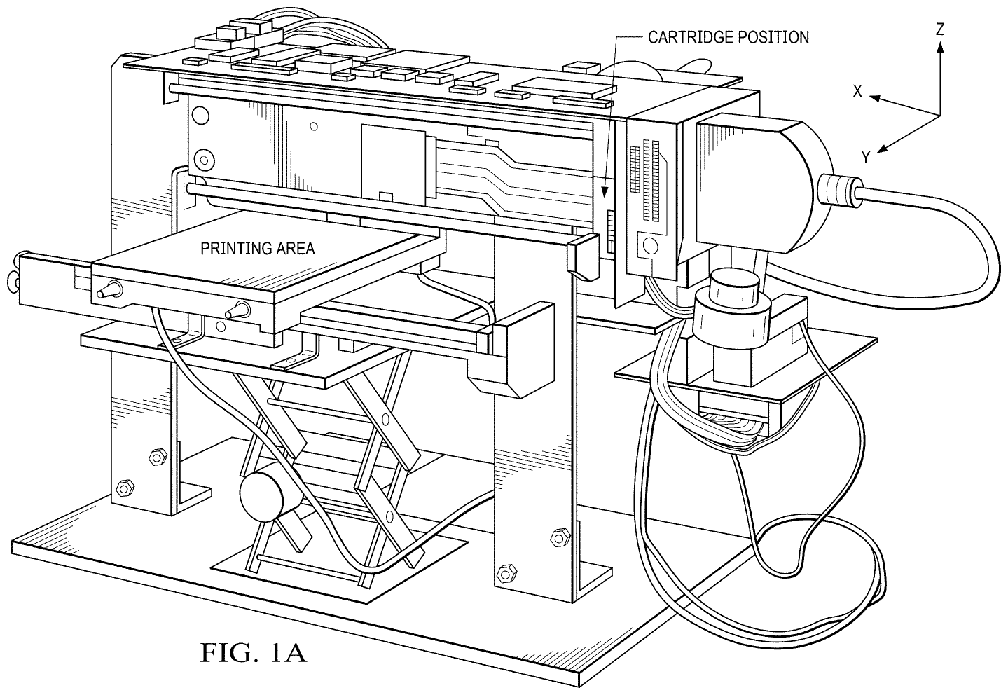

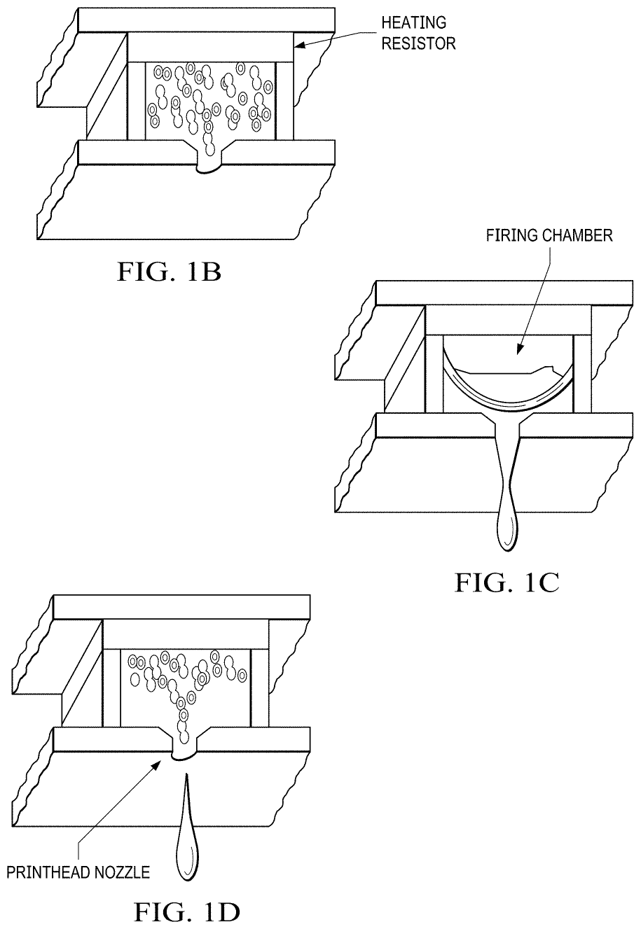

FIG. 1A illustrates a thermal printing apparatus containing generated indued pluripotent stem cells. The induced pluripotent stem cells are generated using a cartridge located at a cartridge position of the thermal printing apparatus. The generated induced pluripotent stem cells are printed within a printing area.

FIGS. 1B-1D illustrates sequential operational schematics of a portion of the thermal printing apparatus that generates the induced pluripotent stem cells. Referring to FIG. 1B, in this thermal printing embodiment, the somatic cells in the cartridge are proximate a heating resistor. Referring to FIG. 1C, the somatic cells are heated within a firing chamber. Referring to FIG. 1D, the somatic cells flow through a printhead nozzle forming at least one droplet. Thus the somatic cells in this thermal printing embodiment are exposed to heat and mechanical stress as they flow in the firing chamber, pass through the printing nozzle and form the droplet. Without being bound by theory, this mechanical stress generates the pluripotent stem cells as demonstrated by some of the cells expressing the Oct-4 gene, which is considered a pluripotent stem cell marker. Of course, the invention is not limited to this embodiment and heating/cooling (thermal stress) is optional.

Methods of operation in accord with embodiments of this disclosure will now be described. A preferred embodiment of this disclosure utilizes a device that generates droplets with a volume of approximately 85 pico liters. However, a range from approximately 30 to approximately 150 pliters can also be generated in accord with alternative embodiments that induce pluripotent stem cells. The droplets are shown schematically in FIGS. 1C-1D. The droplets are a key point of embodiments of this disclosure. While not be bound by theory, without droplet generation, there may not be expression of embryonic stem cell markers. When cells are stretched inside these droplets due to oblong shape of the droplets, the cells will overwhelmingly express Oct-4, Sox-2 and some cells also express Nanog. These three markers are peer review literature accepted as demarking embryonic stem cells. The exact mechanism that causes this expression in adult fibroblasts after droplet formation (e.g. printing) is unclear at this time and under investigation. Nevertheless, embodiments of this disclosure are strong evidence that the cells inside such droplets revert to a more primitive or stem cell-like state.

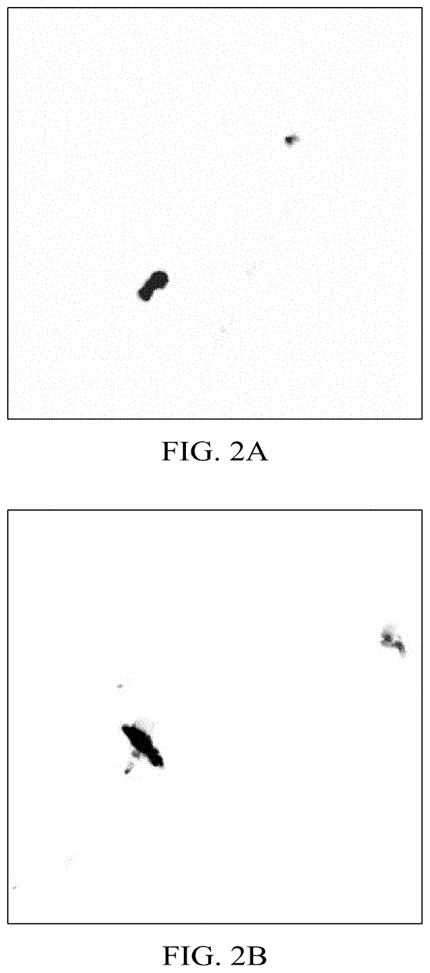

FIGS. 2A-5 show confocal images where the printed cells are stained for expression of Oct-4, Sox-2 and Nanog. FIGS. 2A-2B show cells marked by the fluorescent green stain of the oct-4 antibody labelled to fluoresce green. FIGS. 2A-2B show fibroblasts expressing Oct-4 24 hours after bioprinting. The vividly stained regions show that the embodiment represented by this image works for its intended purpose.

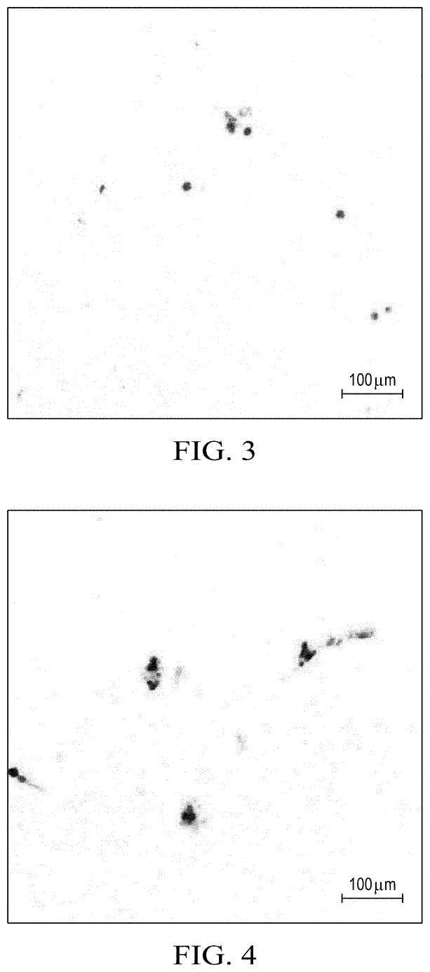

FIG. 3 shows cells stained with a red fluorescent anti-nanog monoclonal antibody. The vividly stained regions show that the embodiment represented by this image works for its intended purpose.

FIG. 4 shows Identical sample as FIG. 3, albeit a different field of view, where cells are stained with anti-Oct-4 monoclonal antibody (green), anti-nano monoclonal antibody (red). Cell nuclei are stained with DAPI (blue). The vividly stained regions show the embodiment represented by this image works for its intended purpose.

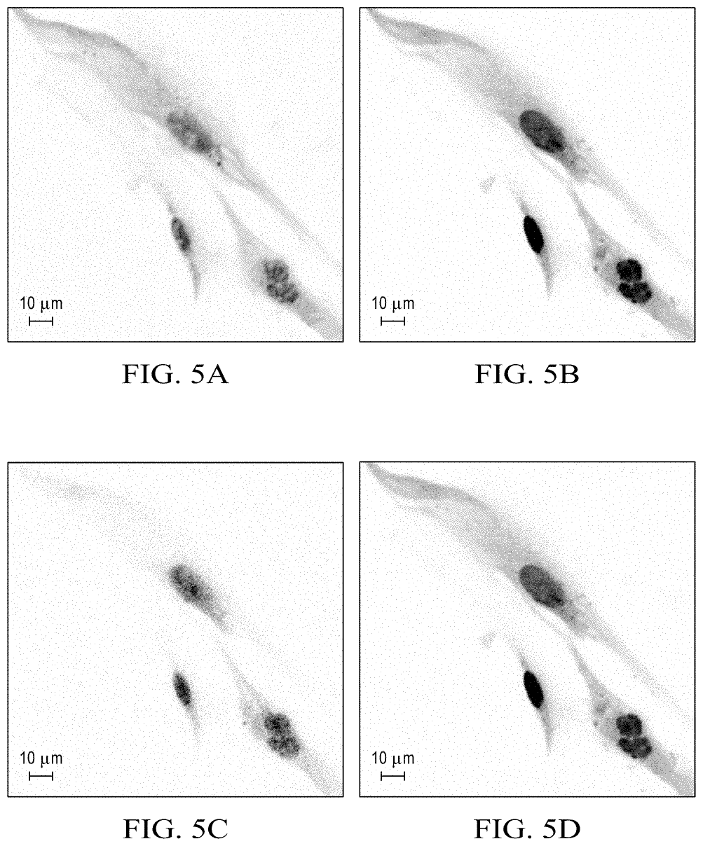

FIGS. 5A-5D show stained cells. FIG. 5A shows the cells with anti-Sox2 monoclonal antibody (red). FIG. 5 B shows the cells with anti-Oct-4 monoclonal antibody (green). FIG. 5C shows the cell nucleus with DAPI (blue). FIG. 5D shows an overlay of A, B and C. The vividly expressed regions show the embodiment represented by this image works for its intended purpose.

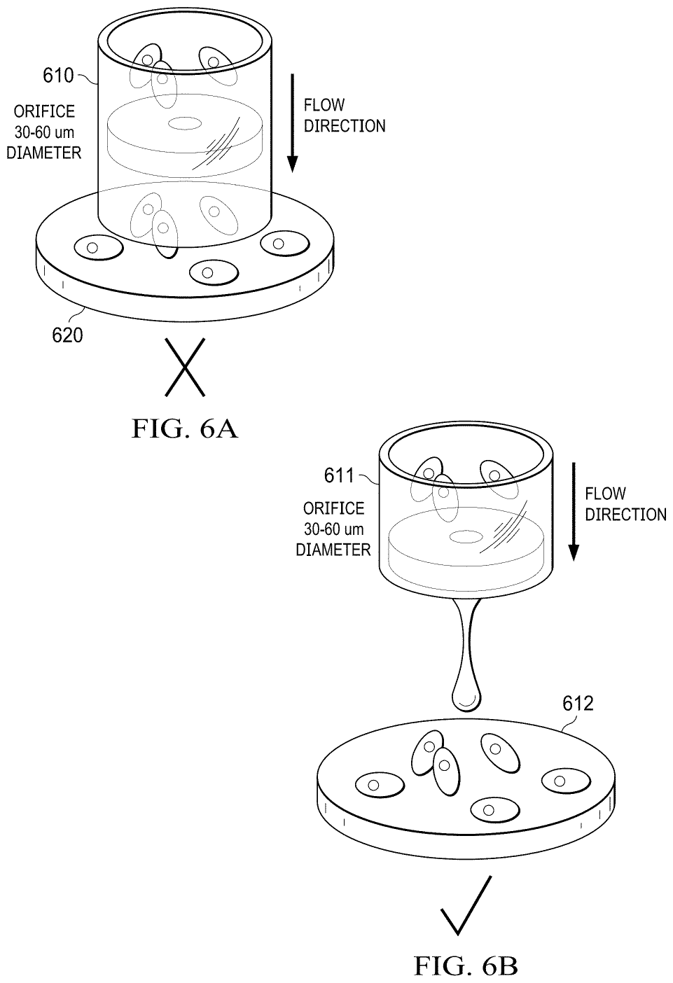

FIGS. 6A-6B show schematics of embodiments of this disclosure in operation. An apparatus can include a columnar constraint 610, 611 and a substrate 620, 612. An orifice with a diameter of from approximately 30 um to approximately 60 um can be advantageously located within the columnar constraint. Referring to FIG. 6A, embodiments of this disclosure may not work well without mechanical stress. Referring to FIG. 6B, preferred embodiment of this disclosure are shown by the advantageous results herein to work well by applying mechanical stress to the somatic cells. The mechanical stress can include shear stress inside a droplet. The mechanical stress can include surface forces (e.g. surface tension) of the droplet causing circulation of the somatic cell within the droplet.

Embodiment of this disclosure can include differentiation of printing induced stem-cells. Applicants have evidence that printed cells form vasculature when incubated with vascular growth factors such as 0.2 ml hydrocortisone; 0.2 ml basic Human fibroblast growth factor-basic (hFGF-B); 0.5 ml vascular endothelial growth factor (VEGF); 0.5 ml of complete human insulin like growth factor-1 with substitution of Arg for Glu³ (R³-IGF-1); 0.5 ml ascorbic acid; 0.5 ml Human epidermal growth factor (hEGF); 0.5 ml gentamicin sulfate-amphotericin (GA-100; and 0.5 ml heparin per 100 ml and fetal bovine serum (FBS).

Printed cells form complete vasculature in animals while pipetted cells form incomplete vasculatures. Applicants have initial evidence that printed fibroblasts can differentiate into cardiomyocytes. This can be confirmed without undue experimentation by analyzing phenotypic expressions.

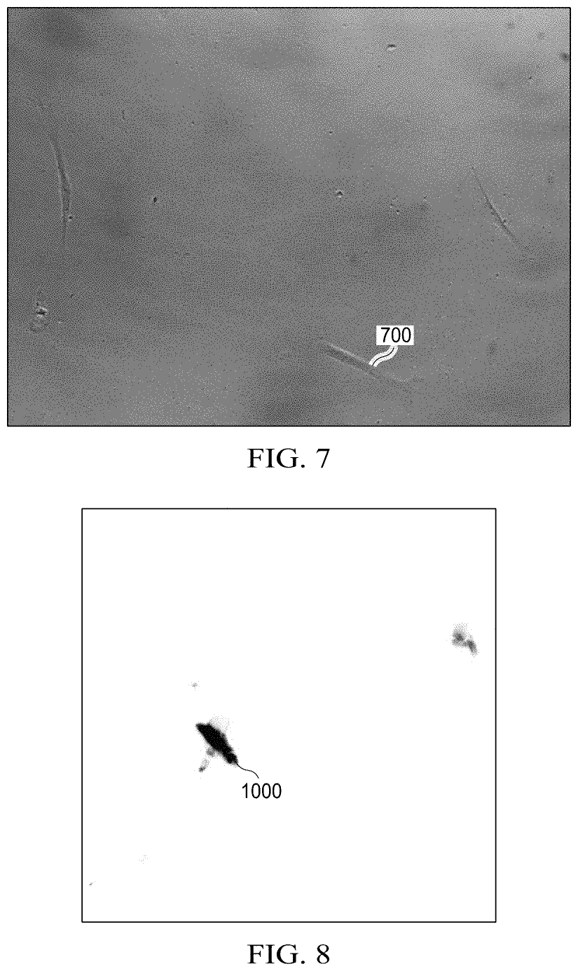

Referring to FIG. 7, morphology of printed cells 700 grown in cardiomyocyte differentiation medium can be appreciated. FIG. 7 shows that the cells appear long and square in shape which is common for adult cardiomyocytes. The cardiomyocytes show that the embodiment represented by this image works for its intended purpose.

Applicants have used 2 sub-generic methods of generating droplets capable of elicitation expression of stem cell markers in fibroblasts. One sub-generic method includes thermal inkjet printing, the other sub-generic method includes pressurized flow through an orifice without heat. However, drops of similar size can also be generated by piezoelectric transducer, laser induced fast forward transfer (LIFT), laser bioprinting (LAB), ultrasonic waves and microvalves. So far, to a first order approximation it seems that the specific method of generating the droplets is not significantly influencing the expression levels of Oct-4, nanog and sox-2 in printed fibroblasts.

Referring to FIG. 8, bioprinted fibroblasts expressing Oct-4 regions 1000 approximately 24 hours after bioprinting are shown. The vividly expressed regions show that the embodiment represented by this image works for its intended purpose.

### Examples

Specific exemplary embodiments will now be further described by the following, nonlimiting examples which will serve to illustrate in some detail various features. The following examples are included to facilitate an understanding of ways in which embodiments of the present disclosure may be practiced. However, it should be appreciated that many changes can be made in the exemplary embodiments which are disclosed while still obtaining like or similar result without departing from the scope of embodiments of the present disclosure. Accordingly, the examples should not be construed as limiting the scope of the present disclosure.

The following list of materials is applicable to both of the examples of stem cell printing protocols described below.
Trypsin/EDTA solution (readily commercially available as Sigma T4049) Dulbecco's Phosphate Buffered Saline (DPBS) (readily commercially available as Sigma D8537)
PluriSTEM™ (readily commercially available as Sigma SCM130) (room temp)
1-1000 ul pipette tips
0-100 ul pipette tips
Well plate or culture ware

Printing of Fibroblasts into Culture Wares

The following example of printing fibroblasts into culture wares should be performed under a biohazard hood.
1. Trypsinize, collect and count fibroblasts (use centrifuge at 1,000 rpm) (see cell passaging protocol).
2. To prepare a 5×10⁵ cells/ml solution, centrifuge cells at 1,000 rpm, aspirate the media and resuspend in the appropriate volume of DPBS.
3. Fill the desired wells of a 96-well plate with 80 μl PluriSTEM™ media.
4. Pipette 100-200 μl of cells in DPBS into the cartridge and print onto the desired wells until empty.
5. Repeat with new cartridge or same cartridge if not clogged until the desired number or amount of cells are printed.
6. Approximate cells/well should be 50,000 in a 96 well plate; 800,000 in a 6-well plate.
7. Alternatively, one can print all cells into one well, collect the cells, optionally centrifuge at 3,000 rpm, count, and divide the cells into wells in the appropriate desired amount. This also allows for seeding cells in ECM coated culture flasks.
8. Incubate at 36° C. and 5% CO₂.
9. Change PluriSTEM™ media after 24 hours, then every other day.

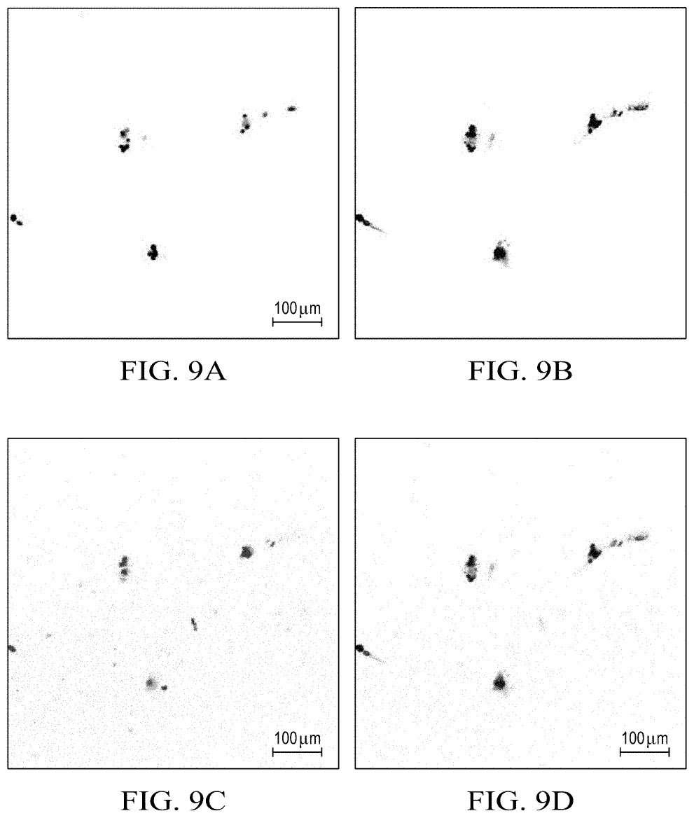

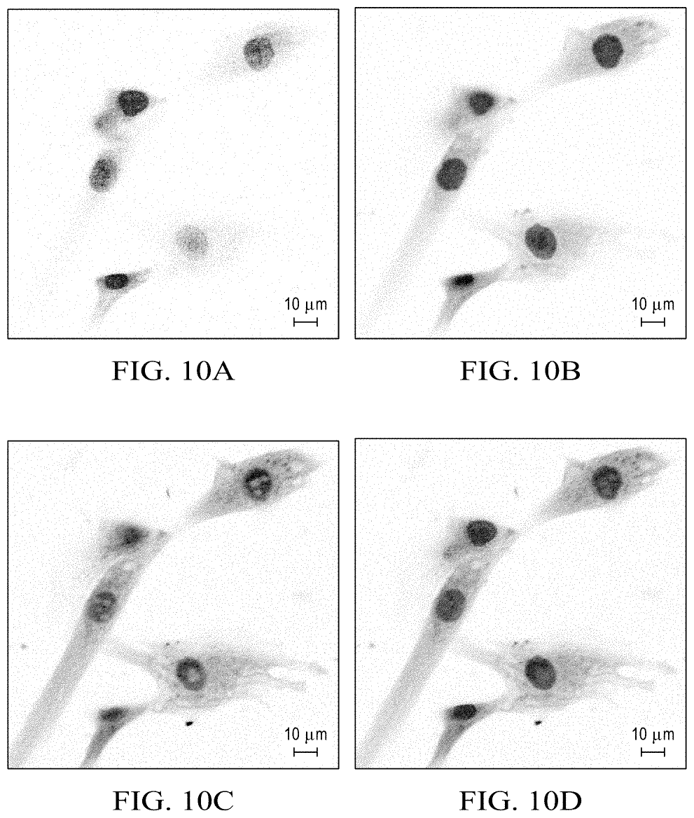

Characterization of the cells printed into wells in the above example was as follows. The cells were stained for 3 pluripotent markers: Oct-4 (green/dark, Nanog and Sox-2 (both red/lighter dark). Blue/medium denotes the cell nucleus. Referring to FIGS. 9A-9D, FIG. 9A shows Nuclei, FIG. 9B shows Oct-4, FIG. 9C shows Nanog, and FIG. 9D shows an overlay of A, B and C. Referring to FIGS. 10A-10D, FIG. 10A shows Nuclei, FIG. 10B shows Oct-4, FIG. 10C shows Sox-2, and FIG. 10D shows an overlay of A, B and C. The clear appearance of the pluripotent markers show that the embodiment represented by this example works for its intended purpose.

Printing of Fibroblasts for Aggregate Formation

The following example of printing fibroblasts for aggregate formation should be performed under a biohazard hood.
1. Follow step 1-5 above.
2. Using an eight channel pipette dispenser (30-300 μl), pipette 40 μl from one column of the 96 well.
3. Lift the lid of a 80.5 mm petri dish, carefully invert it and place it on top of the dish containing 10 ml of PBS.
4. Using the eight-channel pipette, make rows of 40 μl drops on the up-turned inner surface of the lid of the tissue culture dish.
5. Repeat step 2-4 until all the cells are transferred onto the lid in 40 μl drops. Depending on the amount of cells, several petri dish lids may need to be used.
6. Carefully close the lid(s) and place the dish(es) in the incubator for 2 days, with the drops hanging from the top of each lid.
7. After two days, carefully turn over the plate lid, aspirate the drops and put them drops into the well of a 96-well plate.
8. The aggregates are now formed and can be cultured as needed.

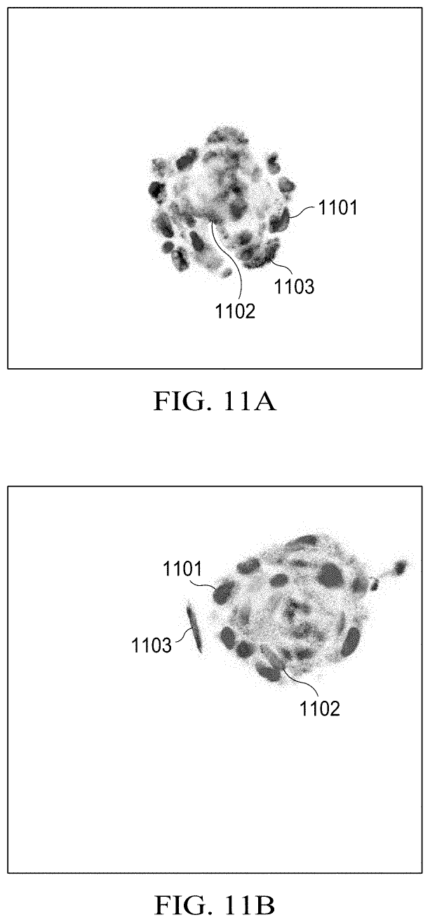

Characterization of the cells aggregated in the above example was as follows. Referring to FIGS. 11A-11B, cells printed into wells and cultured in a hanging drop are shown. The cells were stained for 3 pluripotent markers: Oct-4 (green/dark gray 1103, Nanog and Sox-2 (both red/lighter dark gray 1102). Blue/medium gray 1101 denotes the cell nucleus. The clear appearance of a well-defined aggregate surface shows that the embodiment represented by this example works for its intended purpose.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method of generating induced pluripotent stem cells, comprising:
applying mechanical stress to a somatic cell to produce mechanical strain that induces the somatic cell to become a pluripotent stem cell.

2. The method of claim 1, further comprising forming a droplet that at least partially surrounds the somatic cell before applying.

3. The method of claim 2, wherein forming the droplet includes thermal inkjet printing.

4. The method of claim 2, wherein forming the droplet includes pressurized flow through an orifice without heat.

5. The method of claim 2, wherein forming the droplet includes laser induced fast forward transfer.

6. The method of claim 2, wherein forming the droplet includes using a piezoelectric transducer.

7. The method of claim 2, wherein forming the droplet includes laser bioprinting.

8. The method of claim 2, wherein forming the droplet includes using an ultrasonic transducer.

9. The method of claim 2, wherein forming the droplet includes using at least one microvalve.

10. The method of claim 2, further comprising printing the pluripotent stem cell on a substrate and incubating the pluripotent stem cell.

11. The method of claim 10, further comprising forming an aggregate that comprises the pluripotent stem cell including, after printing and before incubating, positioning the substrate above the pluripotent stem cell whereby gravity exerts a force on the pluripotent stem cell away from the substrate.

12. The method of claim 1, wherein applying mechanical stress includes applying shear stress.

13. The method of claim 2, wherein applying mechanical stress includes surface forces of the droplet causing circulation of the somatic cell within the droplet.

14. The method of claim 1, further comprising heating the somatic cell.

15. A method of generating induced pluripotent stem cells, comprising:
forming a droplet that surrounds a somatic cell; and
applying mechanical stress to the somatic cell to produce mechanical strain that induces the somatic cell to become a pluripotent stem cell without transfection.

16. The method of claim 15, further comprising printing the pluripotent stem cell on a substrate and incubating the pluripotent stem cell.

17. The method of claim 16, further comprising forming an aggregate that comprises the pluripotent stem cell including, after printing and before incubating, positioning the substrate above the pluripotent stem cell whereby gravity exerts a force on the pluripotent stem cell away from the substrate.

18. A method of generating induced pluripotent stem cells, comprising:
forming a droplet that surrounds a somatic cell; and
applying mechanical stress to the somatic cell to produce mechanical strain that induces the somatic cell to become a pluripotent stem cell without transfection,
wherein forming the droplet includes thermal inkjet printing.

19. The method of claim 18, further comprising printing the pluripotent stem cell on a substrate and incubating the pluripotent stem cell.

20. The method of claim 19, further comprising forming an aggregate that comprises the pluripotent stem cell including, after printing and before incubating, positioning the substrate above the pluripotent stem cell whereby gravity exerts a force on the pluripotent stem cell away from the substrate.
